(19) United States
(12) Patent Application Publication
(10) Pub. No.: US 2025/0372044 A1
(43) Pub. Date: Dec. 4, 2025

(54) DISPLAY DEVICE

(71) Applicant: Samsung Display Co., Ltd., Yongin-si (KR)

(72) Inventors: Gyeong Ub MOON, Yongin-si (KR); Yu Na KIM, Yongin-si (KR); Jong Yeop AN, Yongin-si (KR); Dong Wook YANG, Yongin-si (KR); Hyeon Jun LEE, Yongin-si (KR)

(21) Appl. No.: 19/096,524

(22) Filed: Mar. 31, 2025

(30) Foreign Application Priority Data
Jun. 4, 2024 (KR) 10-2024-0073126

(51) Int. Cl. G09G 3/3266

(52) U.S. Cl. CPC G09G 3/3266; G09G 2320/0271; G09G 2360/148; G09G 2380/08

(57) ABSTRACT

A display device includes a display panel, display pixels arranged in a display area of the display panel, light sensing pixels in the display area together with the display pixels, infrared light emitting pixels in the display area together with the display pixels, a display scan driver to drive the display pixels and the light sensing pixels to emit light, a light sensing scan driver to drive the light sensing pixels to detect light, and a main driving circuit to detect pulse wave signals of a user using light sensing signals received through the light sensing pixels and to measure biometric information, wherein the main driving circuit is to separate and generate blood vessel image data from image data and to receive the light sensing signals of a light receiving area by distinguishing the light receiving area according to the blood vessel image data in the display area.

### CROSS-REFERENCE TO RELATED APPLICATION

The present application claims priority to and the benefit of Korean Patent Application No. 10-2024-0073126, filed on Jun. 4, 2024, in the Korean Intellectual Property Office, the entire disclosure of which is incorporated herein by reference.

### BACKGROUND

### 1. Field

Embodiments of the present disclosure relate to a display device.

### 2. Description of the Related Art

As information society develops, there is a growing demand for display devices that display images. Accordingly, display devices are being applied to various electronic devices, such as smartphones, digital cameras, notebook computers, tablet PCs, navigation devices, and smart televisions. Some portable display devices, such as smartphones and tablet PCs, are being equipped with various functions such as image capturing, fingerprint recognition, and/or facial recognition.

With the recent spotlight on the healthcare industry, methods to more easily obtain biometric information, which can provide information about the health of the user, are being developed. For example, attempts are being made to change oscillometric blood pressure measuring devices into portable blood pressure measuring devices. However, portable blood pressure measuring devices require their own independent light source, sensor, and display and can be inconvenient due to having to be carried separately from a portable smartphone or tablet PC.

Recently, efforts have been made to combine portable display devices such as smartphones and tablet PCs with portable blood pressure measuring devices. However, it has been difficult to secure conditions for measuring biometric information, such as signal-to-noise ratio (SNR), due to limitations resulting from the amount of light emission or luminance of the display device displaying the images, the lifespan of a light source, reliability, and/or the like.

The above information disclosed in this Background section is intended to enhance understanding of the background of the disclosure and may contain information that does not constitute prior art.

### SUMMARY

Aspects of one or more embodiments of the present disclosure are directed to a display device which can detect a photoplethysmography signal using an image display panel and measure a user's biometric information such as blood pressure.

In addition, aspects of one or more embodiments of the present disclosure are directed to a display device which can set a user's blood vessel placement pattern and a corresponding area by analyzing an image of the user's touched body part and detect a bio-signal, such as a pulse wave signal, through the blood vessel placement pattern area.

However, aspects of the present disclosure are not restricted to the ones set forth herein. Additional aspects will be set forth in part in the description which follows and, in part, will be apparent from the description, or may be learned by practice of the presented embodiments of the disclosure.

According to an one or more embodiments of the present disclosure, a display device includes a display panel; display pixels arranged in a display area of the display panel, light sensing pixels arranged in the display area together with the display pixels, infrared light emitting pixels arranged in the display area together with the display pixels, a display scan driver to drive the display pixels and the light sensing pixels to emit light, a light sensing scan driver to drive the light sensing pixels to detect light, and a main driving circuit to detect pulse wave signals of a user using light sensing signals received through the light sensing pixels and measuring biometric information, wherein the main driving circuit separates and generates (e.g., is configured to separate and generate) blood vessel image data from image data for a touch area during a user's touch position detection period and receives (e.g., to receive) the light sensing signals of a light receiving area by distinguishing the light receiving area according to the blood vessel image data in the display area during a user's biometric information detection period.

According to one or more embodiments of the present disclosure, a display device includes a display panel; display pixels arranged in a display area of the display panel, light sensing pixels arranged in the display area together with the display pixels, infrared light emitting pixels arranged in the display area together with the display pixels, a display scan driver to drive the display pixels and the light sensing pixels to emit light, a light sensing scan driver to drive the light sensing pixels to detect light, and a main driving circuit to detect pulse wave signals of a user using light sensing signals received through the light sensing pixels and to measure biometric information, wherein the main driving circuit displays (e.g., is configured to display) a biometric information measurement area as an application program screen in the display area, drives (e.g., to drive) the display pixels or the infrared light emitting pixels arranged in the biometric information measurement area by controlling a scan signal output of the display scan driver and the light sensing scan driver, and receives (e.g., to receive) the light sensing signals through the light sensing pixels of the biometric information measurement area.

In a display device according to one or more embodiments, if (e.g., when) light emitted from an image display pixel is reflected by a specific body part such as a user's finger, the reflected light may be detected by a light sensing pixel of a display panel to measure the user's biometric information, such as blood pressure. Accordingly, the user's biometric information, such as blood pressure, can be detected using the display panel of the display device.

A display device according to one or more embodiments can set a user's blood vessel placement pattern and a corresponding area by analyzing an image of the user's touched body part and can detect a bio-signal, such as a pulse wave signal, through the blood vessel placement pattern area. Accordingly, the user can be recognized through the analysis result of the blood vessel placement pattern area, and the user's biometric information can be measured more accurately.

However, the features and effects of the present disclosure are not restricted to the ones set forth herein. The above and other features and effects of the present disclosure will become more apparent to one of daily skill in the art to which the present disclosure pertains by referencing the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and/or other aspects, features and/or principles will become apparent and more readily appreciated from the following description of one or more embodiments of the present disclosure, taken in conjunction with the accompanying drawings, in which:

FIG. 1 is a perspective view of a display device according to one or more embodiments of the present disclosure;

FIG. 2 is a plan view illustrating the arrangement structure of a display panel and a display driving circuit of FIG. 1, according to one or more embodiments of the present disclosure;

FIG. 3 is a plan view illustrating an arrangement structure of a display panel and a display driving circuit according to one or more embodiments of the present disclosure;

FIG. 4 is a side view illustrating a configuration of the display device of FIGS. 1 and 2, according to one or more embodiments of the present disclosure;

FIG. 5 is a side view illustrating a configuration of the display device of FIGS. 1 and 2, according to one or more embodiments of the present disclosure;

FIG. 6 is a schematic layout view of the display panel illustrated in FIGS. 1 through 4, according to one or more embodiments of the present disclosure;

FIG. 7 is a layout view of a display area according to one or more embodiments of the present disclosure;

FIG. 8 is a circuit diagram of a display pixel and a light sensing pixel according to one or more embodiments of the present disclosure;

FIG. 9 is a flowchart illustrating a biometric information extraction process of a main driving circuit according to one or more embodiments of the present disclosure;

FIG. 10 shows images illustrating a pulse wave detection pattern area setting method of the main driving circuit according to one or more embodiments of the present disclosure;

FIG. 11 shows images illustrating a method of setting an emission driving area (e.g., a light emitting area) and a light sensing area (e.g., a light receiving area) separately in the pulse wave detection pattern area illustrated in FIG. 10, according to one or more embodiments of the present disclosure;

FIG. 12 is a diagram illustrating a reflected light sensing area and a pulse wave detection pattern area distinguished in a display area of one or more embodiments of the present disclosure;

FIG. 13 is a flowchart illustrating a biometric information extraction process of the main driving circuit according to one or more embodiments of the present disclosure;

FIG. 14 shows images illustrating a pulse wave detection pattern area setting method of the main driving circuit according to one or more embodiments of the present disclosure;

FIG. 15 is a diagram illustrating a user recognition method using a pulse wave detection pattern area, according to one or more embodiments of the present disclosure;

FIG. 16 is a graph for explaining a method of calculating blood pressure information from among biometric information, according to one or more embodiments of the present disclosure;

FIG. 17 is a graph for explaining a method of calculating information about heartbeat and respiration from among the biometric information, according to one or more embodiments of the present disclosure;

FIG. 18 is a graph for explaining a method of calculating information about blood vessel elasticity from among the biometric information, according to one or more embodiments of the present disclosure;

FIG. 19 is a graph for explaining a method of calculating information about cardiovascular disease from among the biometric information, according to one or more embodiments of the present disclosure;

FIG. 20 is a graph for explaining a method of calculating information about oxygen saturation from among the biometric information, according to one or more embodiments of the present disclosure; and

FIG. 21 illustrates the results of measuring biometric information using a display device, according to one or more embodiments of the present disclosure.

### DETAILED DESCRIPTION

The present disclosure may be modified in many alternate forms, and thus specific embodiments will be illustrated in the drawings and described in more detail. It should be understood, however, that this is not intended to limit the present disclosure to the particular forms disclosed, but rather, is intended to cover all modifications, equivalents, and alternatives falling within the spirit and scope of the present disclosure.

The present disclosure will now be described more fully hereinafter with reference to the accompanying drawings. This disclosure may, however, be embodied in different forms and should not be construed as limited to the illustrated embodiments set forth herein. Rather, these embodiments are provided so that this disclosure will be thorough and complete, and will fully convey the aspects and features of the present disclosure to those skilled in the art. Accordingly, processes, elements, and techniques that are not necessary to those having ordinary skill in the art for a complete understanding of the aspects and features of the present disclosure may not be described.

It will be understood that when an element, such as an area, layer, film, region or portion, is referred to as being “on” or “connected to” another element, it can be directly on or connected to the other element, or one or more intervening elements may be present. In contrast, when an element or layer is referred to as being “directly on,” “directly connected to”, or “immediately adjacent to” another element or layer, there are no intervening elements or layers present. In addition, it will also be understood that when an element is referred to as being “between” two elements, it can be the only element between the two elements, or one or more intervening elements may also be present.

Unless otherwise noted, like reference numerals denote like elements throughout the attached drawings and the written description, and thus, duplicative descriptions thereof may not be provided.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

Spatially relative terms, such as “on,” “below,” “lower,” “under,” “above,” “upper,” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the drawings. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

As used herein, the singular forms “a,” “an” and “the” are intended to include the plural forms as well, unless the context clearly indicates otherwise. It will be further understood that the terms “comprises,” “comprising,” “includes,” “including,” “have,” and “having,” when used in this specification, specify the presence of the stated features, integers, steps, operations, elements, and/or components, but do not preclude the presence or addition of one or more other features, integers, steps, operations, elements, components, and/or groups thereof.

As used herein, the term “and/or” includes any and all combinations of one or more of the associated listed items. Unless otherwise apparent from the disclosure, expressions such as “at least one of,” “a plurality of,” “one of,” and other prepositional phrases, when preceding a list of elements, should be understood as including the disjunctive if written as a conjunctive list and vice versa. For example, the expressions “at least one of a, b, or c,” “at least one of a, b, and/or c,” “one selected from the group consisting of a, b, and c,” “at least one selected from among a, b, and c,” “at least one from among a, b, and c,” “one from among a, b, and c”, “at least one of a to c” indicates only a, only b, only c, both a and b, both a and c, both b and c, all of a, b, and c, or variations thereof.

In the drawings, the relative sizes of elements, layers, and regions may be exaggerated for clarity.

As used herein, the terms “use,” “using,” and “used” may be considered synonymous with the terms “utilize,” “utilizing,” and “utilized,” respectively.

Each of the features of the various embodiments of the present disclosure may be combined or combined with each other, in part or in whole, and various interlocking and driving are possible. Each embodiment may be implemented independently of each other or may be implemented together in an association.

In the context of the present disclosure and unless otherwise defined, a plan view is an orthographic projection of a three-dimensional object from the position of a horizontal plane through the object. That is, it is a top-down view, showing the layout and spatial relationships of various elements within the object or structure. A plan view based on the direction DR3 refers to a top-down view of the display panel, as if looking directly down onto the surface from above. In this context, DR3 is the direction perpendicular or normal to the plane defined by the first direction (DR1) and the second direction (DR2). This refers to that in a plan view, the arrangement of sub-pixels, pads, and other components as they are laid out on the substrate can be seen, without any perspective distortion.

Hereinafter, one or more embodiments will be described with reference to the attached drawings.

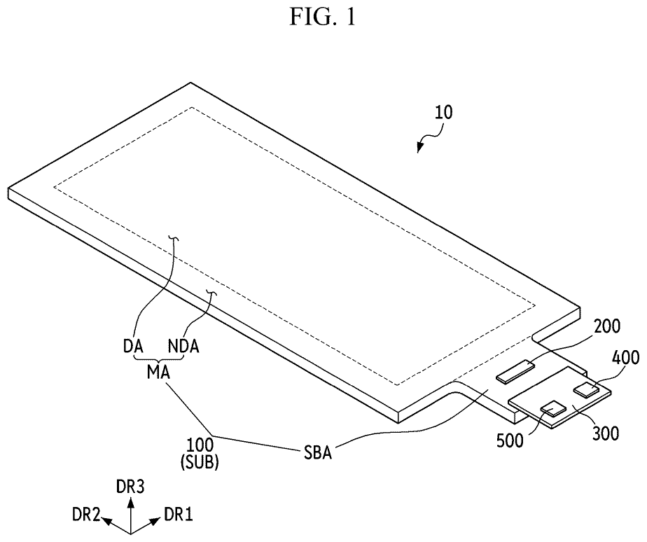

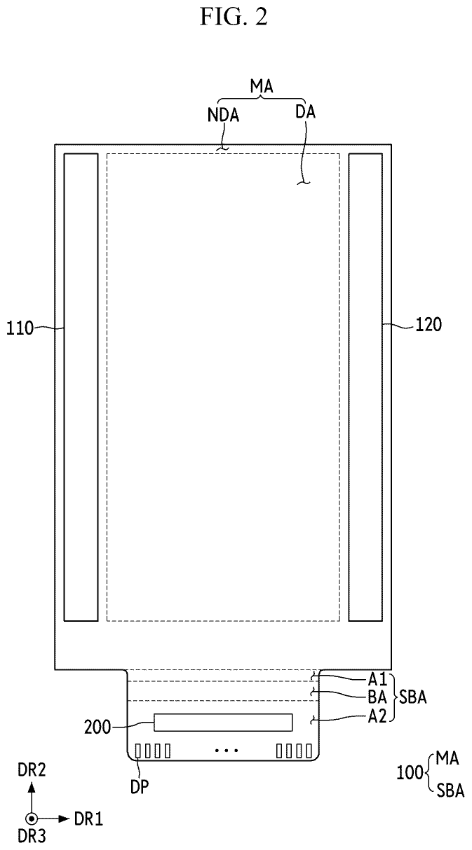

FIG. 1 is a perspective view of a display device 10 according to one or more embodiments of the present disclosure. FIG. 2 is a plan view illustrating an arrangement structure of a display panel 100 and a display driving circuit illustrated in FIG. 1, according to one or more embodiments of the present disclosure.

Referring to FIGS. 1 and 2, the display device 10 according to one or more embodiments may be applied to portable electronic devices such as mobile phones, smartphones, tablet personal computers (PCs), mobile communication terminals, electronic notebooks, electronic books, portable multimedia players (PMPs), navigation devices, and/or ultra-mobile PCs (UMPCs). Alternatively, the display device 10 according to one or more embodiments may be applied as a display unit of a television, a notebook computer, a monitor, a billboard, and/or an Internet of things (IoT) device. Alternatively, the display device 10 according to one or more embodiments may be applied to wearable devices such as smart watches, watch phones, glasses-type (kind) displays, and/or head-mounted displays (HMDs). Alternatively, the display device 10 according to one or more embodiments may be applied to a car display, such as a display in a dashboard of a vehicle, a center fascia of a vehicle, a center information display (CID) arranged on a dashboard of a vehicle, a room mirror display replacing side mirrors of a vehicle, and/or a display arranged on the back of a front seat as an entertainment for rear-seat passengers of a vehicle.

The display device 10 may be a light emitting display device such as an organic light emitting display device using an organic light emitting diode, a quantum dot light emitting display device including a quantum dot light emitting layer, an inorganic light emitting display device including an inorganic semiconductor, or a micro- or nano-light emitting display device using a micro- or nano-light emitting diode. Embodiments in which the display device 10 is an organic light emitting display device will be mainly described in more detail below, but the present disclosure is not limited thereto.

The display panel 100 may be formed in a rectangular planar shape having short sides in a first direction DR1 and long sides in a second direction DR2 intersecting the first direction DR1. Each corner where a short side extending in the first direction DR1 meets a long side extending in the second direction DR2 may be right-angled or may be rounded to have a set or predetermined curvature. The planar shape of the display panel 100 is not limited to a quadrangular shape but may also be other polygonal shapes, a circular shape, or an elliptical shape. The display panel 100 may be formed flat, but the present disclosure is not limited thereto. For example, the display panel 100 may include a curved portion formed at left and right ends and having a constant or varying curvature. In one or more embodiments, the display panel 100 may be formed to be flexible so that it can be curved, bent, folded, or rolled.

A substrate SUB of the display panel 100 may be divided into a main area MA and a sub-area SBA.

The main area MA may be divided into a display area DA which displays an image and a non-display area NDA arranged around the display area DA.

The non-display area NDA may neighbor the display area DA. The non-display area NDA may be an area outside the display area DA. The non-display area NDA may be around (e.g., surround) the display area DA. The non-display area NDA may be an edge area of the display panel 100.

The display area DA includes display pixels which display an image and light sensing pixels which detect light reflected from a front protective cover or protective glass and a user's body part such as a finger. The display area DA may occupy most of the main area MA. The display area DA may be arranged in a center of the main area MA.

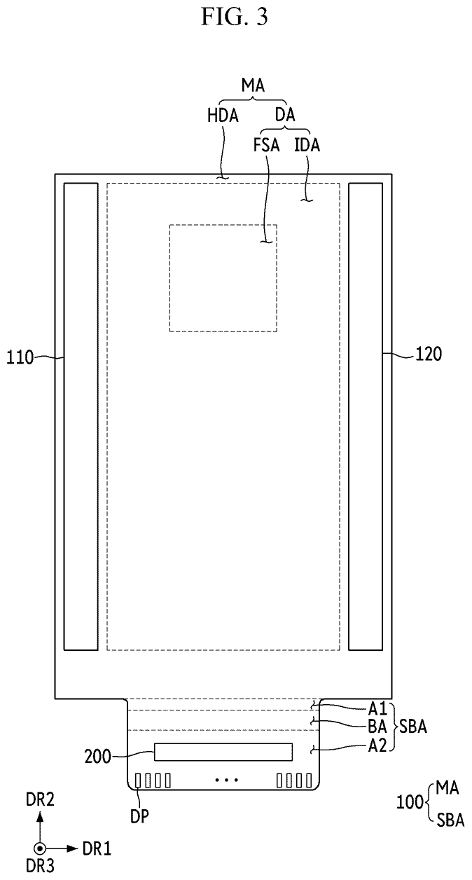

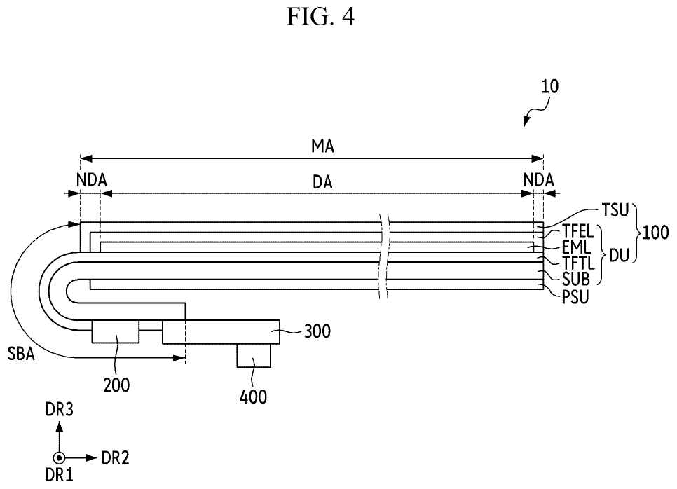

FIG. 3 is a plan view illustrating an arrangement structure of a display panel 100 and a display driving circuit according to one or more embodiments of the present disclosure. FIG. 4 is a side view illustrating the configuration of the display device 10 illustrated in FIGS. 1 and 2, according to one or more embodiments of the present disclosure.

Referring to FIGS. 1, 3, and 4, the display device 10 includes the display panel 100, a main driving circuit 200, a touch sensing unit TSU, a pressure sensing unit PSU, a circuit board 300, a touch driving circuit 400, and a biometric information storage memory 500.

Referring to FIG. 3, the display panel 100 may be formed in a rectangular planar shape having short sides in the first direction DR1 and long sides in the second direction DR2 intersecting the first direction DR1. Each corner where a short side extending in the first direction DR1 meets a long side extending in the second direction DR2 may be right-angled or may be rounded to have a set or predetermined curvature. The planar shape of the display panel 100 is not limited to a quadrangular shape but may also be other polygonal shapes, a circular shape, or an elliptical shape. The display panel 100 may be formed flat, but the present disclosure is not limited thereto. For example, the display panel 100 may include a curved portion formed at left and right ends and having a constant or varying curvature. In one or more embodiments, the display panel 100 may be formed to be flexible so that it can be curved, bent, folded, or rolled.

Referring to FIG. 3, a display area DA may be divided into an image display area IDA where only display pixels are arranged without light sensing pixels and a biometric information measurement area FSA where both display pixels and light sensing pixels are arranged to detect bioinformation including a pulse wave signal. For example, light sensing pixels formed to detect a pulse wave signal may be arranged together with display pixels only in the biometric information measurement area FSA in a preset part of the display area DA of the display panel 100.

A sub-area SBA may protrude from a side of a main area MA in the second direction DR2. A length of the sub-area SBA in the second direction DR2 may be smaller than a length of the main area MA in the second direction DR2. A length of the sub-area SBA in the first direction DR1 may be smaller than a length of the main area MA in the first direction DR1 or may be substantially equal to the length of the main area MA in the first direction DR1.

The sub-area SBA may include a first area A1, a second area A2, and a bending area BA.

The first area A1 is an area protruding from a side of the main area MA in the second direction DR2. A side of the first area A1 may contact a non-display area NDA of the main area MA, and the other side of the first area A1 may contact the bending area BA.

The second area A2 is an area where pads DP and the main driving circuit 200 are arranged. The main driving circuit 200 may be attached to driving pads of the second area A2 using a conductive adhesive member such as an anisotropic conductive layer. The circuit board 300 may be attached to the pads DP of the second area A2 using a conductive adhesive member. A side of the second area A2 may contact the bending area BA.

The bending area BA is a bendable area. When the bending area BA is bent, the second area A2 may be placed below the first area A1 and below the main area MA. The bending area BA may be arranged between the first area A1 and the second area A2. A side of the bending area BA may contact the first area A1, and the other side of the bending area BA may contact the second area A2.

As illustrated in FIG. 4, the sub-area SBA may be bent. In such embodiments, the sub-area SBA may be placed below the main area MA. The sub-area SBA may overlap the main area MA in a third direction DR3.

The touch sensing unit TSU which detects a touch position of a body part such as a finger may be arranged on the front of the display panel 100 including the display area DA. The touch sensing unit TSU may include a plurality of touch electrodes to detect a user's touch in a capacitive manner.

The touch sensing unit TSU includes a plurality of touch electrodes arranged to intersect each other in the first and second directions DR1 and DR2. For example, the touch electrodes include a plurality of driving electrodes spaced and/or apart (e.g., spaced apart or separated) and arranged side by side in the first direction DR1 and a plurality of sensing electrodes spaced and/or apart (e.g., spaced apart or separated) and arranged side by side in the second direction DR2 to intersect the driving electrodes with an organic material layer or an inorganic material layer interposed between them. The driving electrodes and the sensing electrodes may be formed to extend in a wiring area between display pixels SP and light sensing pixels LSP arranged in the display area DA so as not to overlap the display pixels SP and the light sensing pixels LSP. The driving electrodes and the sensing electrodes form mutual capacitance and transmit touch sensing signals, which vary according to a user's touch, to the touch driving circuit 400.

The touch driving circuit 400 may detect a change in mutual capacitance between the touch electrodes input from the touch electrodes and supply touch data according to the change in capacitance and coordinate data of a touch-detected position to the main driving circuit 200.

The touch driving circuit 400 supplies touch driving signals to the driving electrodes, respectively, and receives touch sensing signals from the sensing electrodes RE, respectively. In one or more embodiments, the touch driving circuit 400 detects a change in mutual capacitance between the driving electrodes and the sensing electrodes according to a change in the size of the touch sensing signals. The touch driving circuit 400 generates touch data according to the change in mutual capacitance between the driving electrodes and the sensing electrodes and derives a touch-detected position. Accordingly, coordinate data of the touch-detected position may be supplied to the main driving circuit 200.

The pressure sensing unit PSU which detects pressure applied by a body part such as a finger may be arranged or formed on a back surface of the display panel 100, for example, a back surface of the substrate SUB. The pressure sensing unit PSU may be formed as a transparent sheet type (kind) in which a plurality of transparent electrodes are arranged in vertical and horizontal directions and may be arranged in the entire main area MA. In one or more embodiments, the pressure sensing unit PSU may be arranged or formed on the inside or the front of the display panel 100.

For example, the pressure sensing unit PSU includes a plurality of pressure sensing electrodes arranged to intersect each other in the first and second directions DR1 and DR2. The pressure sensing electrodes include a plurality of lower electrodes spaced and/or apart (e.g., spaced apart or separated) and arranged side by side in the first direction DR1 and a plurality of upper electrodes spaced and/or apart (e.g., spaced apart or separated) and arranged side by side in the second direction DR2 to intersect the lower electrodes with a transparent inorganic (or organic) material layer interposed between them. The lower electrodes and the upper electrodes form self-capacitance with the transparent inorganic (or organic) material layer interposed between them and transmit pressure sensing signals, which vary according to a user's touch pressure, to the touch driving circuit 400.

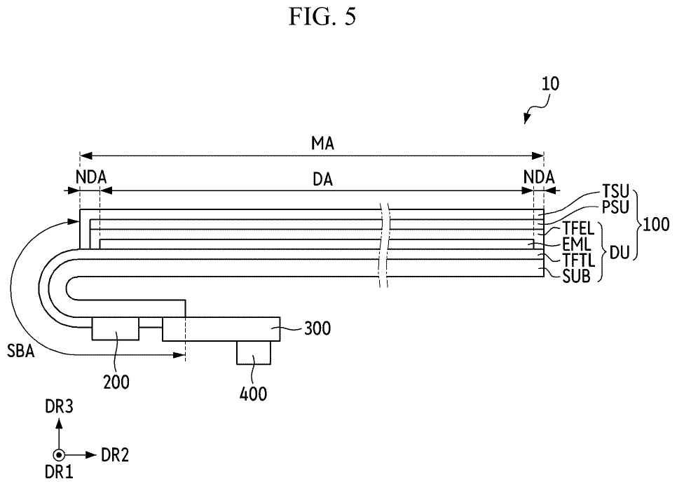

FIG. 5 is a side view of illustrating a configuration of the display device 10 illustrated in FIGS. 1 and 2, according to one or more embodiments of the present disclosure.

FIG. 5 illustrates an example in which the pressure sensing unit PSU is arranged on a front surface of the display panel 100, for example, on a surface between the display panel 100 and the touch sensing unit TSU.

When the pressure sensing unit PSU is arranged on the front surface of the display panel 100, the pressure sensing electrodes of the pressure sensing unit PSU, i.e., a plurality of lower electrodes and upper electrodes may be formed to extend in a wiring area between display pixels and light sensing pixels arranged in the display area DA so as not to overlap the display pixels and the light sensing pixels. The touch driving circuit 400 may supply a reference voltage to the lower electrodes of the pressure sensing unit PSU and receive pressure sensing signals from the upper electrodes to detect a change in self-capacitance of pressed areas through the pressure sensing signals. Accordingly, the touch driving circuit 400 may generate pressure data according to the amount of change in self-capacitance and detection coordinate data of a pressure-detected position and supply the pressure data to the main driving circuit 200. The pressure sensing unit PSU may also be applied in one or more suitable structures other than the structure using pressure sensing electrodes and is not limited to the description of FIGS. 3 and 4.

The circuit board 300 may be attached to an end of the sub-area SBA. Accordingly, the circuit board 300 may be electrically connected to the display panel 100 and the main driving circuit 200. The display panel 100 and the main driving circuit 200 may receive digital video data, timing signals, and driving voltages through the circuit board 300. The circuit board 300 may be a flexible printed circuit board, a printed circuit board, or a flexible film such as a chip on film.

The main driving circuit 200 may generate electrical signals such as control signals and data voltages for driving the display panel 100. Each of the main driving circuit 200 and the touch driving circuit 400 may be formed as an integrated circuit and attached onto the display panel 100 or the circuit board 300 using a chip on glass (COG) method, a chip on plastic (COP) method, or an ultrasonic bonding method. However, the present disclosure is not limited thereto. For example, each of the main driving circuit 200 and the touch driving circuit 400 may also be attached onto the circuit board 300 using a chip on film (COF) method.

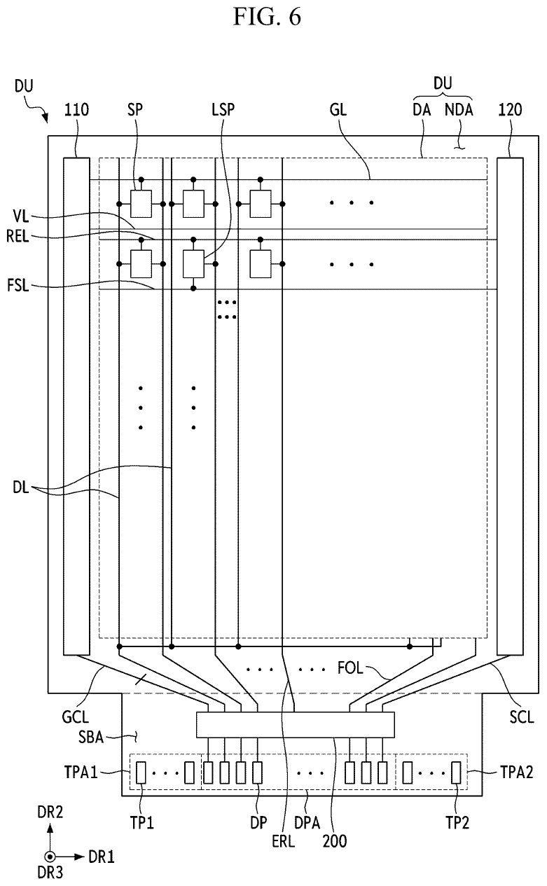

FIG. 6 is a schematic layout view of the display panel 100 illustrated in FIGS. 1 through 4, according to one or more embodiments of the present disclosure. For example, FIG. 6 is a layout view illustrating the display area DA and the non-display area NDA of a display module DU before the touch sensing unit TSU is formed. Referring to FIG. 6 together with FIGS. 3 and 4, a display scan driver 110, a

light sensing scan driver 120, and the main driving circuit 200 may be arranged on the display panel 100 of the display device 10 according to one or more embodiments. In one or more embodiments, the touch driving circuit 400 and a power supply unit may be arranged on the circuit board 300 connected to the display panel 100. Here, both the main driving circuit 200 and the touch driving circuit 400 may be integrally formed as a 1-chip type (kind) and mounted on the display panel 100 or the circuit board 300. However, for ease of functional description, an example in which the main driving circuit 200 and the touch driving circuit 400 are formed as different integrated circuits will be described in more detail below.

Referring to FIG. 6, the display panel 100 may include display pixels SP, infrared light emitting pixels, light sensing pixels LSP, display scan lines GL, emission control lines VL, data lines DL, light sensing scan lines FSL, sensing reset lines REL, and light sensing lines ERL arranged in the display area DA. The display scan driver 110 and the light sensing scan driver 120 are arranged in the non-display area NDA.

The display scan lines GL sequentially supply display scan signals received from the display scan driver 110 on a horizontal line-by-horizontal line basis to each of the display pixels SP, the light sensing pixels LSP, and the infrared light emitting pixels ISP (e.g., each of the display pixels SP, the light sensing pixels LSP, and the infrared light emitting pixels ISP is connected to a horizontal line of the display scan lines GL). The display scan lines GL may extend in the first direction DR1 and may be spaced and/or apart (e.g., spaced apart or separated) from each other in the second direction DR2 intersecting the first direction DR1.

The emission control lines VL sequentially supply emission control signals received from the display scan driver 110 on a horizontal line-by-horizontal line basis to each of the display pixels SP, the light sensing pixels LSP and the infrared light emitting pixels ISP (e.g., each of the display pixels SP, the light sensing pixels LSP, and the infrared light emitting pixels ISP is connected to a horizontal line of the emission control lines VL). The emission control lines VL may extend parallel to the display scan lines GL in the first direction DR1 and may be spaced and/or apart (e.g., spaced apart or separated) from each other in the second direction DR2 intersecting the first direction DR1.

The data lines DL may supply data voltages received from the main driving circuit 200 to a plurality of display pixels SP. The data lines DL may extend in the second direction DR2 and may be spaced and/or apart (e.g., spaced apart or separated) from each other in the first direction DR1.

The light sensing scan lines FSL sequentially supply sensing scan signals received from the light sensing scan driver 120 on a horizontal line-by-horizontal line basis to the plurality of light sensing pixels LSP (e.g., each of the light sensing pixels LSP is connected to a horizontal line of the light sensing scan lines FSL). The light sensing scan lines FSL may extend in the first direction DR1 and may be spaced and/or apart (e.g., spaced apart or separated) from each other in the second direction DR2 intersecting the first direction DR1.

The sensing reset lines REL sequentially supply sensing reset signals received from the light sensing scan driver 120 on a horizontal line-by-horizontal line basis to the plurality of light sensing pixels LSP (e.g., each of the light sensing pixels LSP is connected to a horizontal line of the sensing reset lines REL). The sensing reset lines REL may extend parallel to the light sensing scan lines FSL in the first direction DR1 and may be spaced and/or apart (e.g., spaced apart or separated) from each other in the second direction DR2 intersecting the first direction DR1.

The light sensing lines ERL are connected between the light sensing pixels LSP and the main driving circuit 200 to supply light sensing signals respectively output from the light sensing pixel LSP to the main driving circuit 200. The light sensing lines ERL may lie and extend in the second direction DR2 according to the direction in which the main driving circuit 200 is arranged and may be spaced and/or apart (e.g., spaced apart or separated) from each other in the first direction DR1.

The non-display area NDA may be around (e.g., surround) the display area DA. The non-display area NDA may include the display scan driver 110, the light sensing scan driver 120, fan-out lines FOL, gate control lines GCL, and light sensing control lines SCL.

Display pixels SP and a light sensing pixel LSP may form a first unit pixel (e.g., each first unit pixel may include display pixels SP and a light sensing pixel LSP) and the first unit pixels may be arranged in a matrix form in the first direction DR1 and the second direction DR2 in the display area DA. In one or more embodiments, display pixels SP and an infrared light emitting pixel ISP may form a second unit pixel (e.g., each second unit pixel may include display pixels SP and an infrared light emitting pixel ISP), and the second unit pixels may be arranged alternately with the first unit pixels in a matrix form in the display area DA.

For example, three display pixels SP respectively displaying red light, green light and blue light and one light sensing pixel LSP may form each first unit pixel. In one or more embodiments, three display pixels SP respectively displaying red light, green light and blue light and one infrared light emitting pixel ISP may form each second unit pixel. The first unit pixels and the second unit pixels may be arranged in a matrix form by alternating in a horizontal or vertical stripe form. In one or more embodiments, the first unit pixels and the second unit pixels may be alternately arranged in a zigzag form in plan view and may be arranged in a matrix form in any one diagonal direction.

Each of the red, green and blue display pixels SP and the infrared light emitting pixels ISP may be connected to any one of the display scan lines GL and any one of the emission control lines VL. During an image display period, each of the red, green and blue display pixels SP may receive a data voltage of a data line DL according to a display scan signal of a display scan line GL and an emission control signal of an emission control line VL and may be to emit light by supplying a driving current to a light emitting element according to the data voltage. Here, during a period of measuring biometric information such as blood pressure, each of display pixels SP which display at least one color among the red, green and blue display pixels SP may display light by optionally receiving a data voltage for light emission, together with a display scan signal and an emission control signal. In one or more embodiments, during the period of measuring biometric information such as blood pressure, each of the infrared light emitting pixels ISP may display infrared light by optionally receiving a data voltage for light emission, together with a display scan signal and an emission control signal.

The light sensing pixels LSP may be arranged alternately with the red, green and blue display pixels SP in the vertical or horizontal direction. Each of the light sensing pixels LSP may be connected to one of the light sensing scan lines FSL, one of the sensing reset lines REL, and one of the light sensing lines ERL. During the period of measuring biometric information such as blood pressure, each of the light sensing pixels LSP may be reset in response to a sensing reset signal from a sensing reset line REL and may generate a light sensing signal corresponding to the amount of reflected light incident from the front side. In one or more embodiments, each of the light sensing pixels LSP may be to transmit the light sensing signal to a light sensing line ERL in response to a sensing scan signal from a light sensing scan line FSL. In one or more embodiments, each of the light sensing pixels LSP may be

connected to one of the display scan lines GL on a horizontal line-by-horizontal line basis. Each of the light sensing pixels LSP may generate a light sensing signal corresponding to the amount of reflected light incident from the front side and may output the light sensing signal to a light sensing line ERL in response to a display scan signal received through a display scan line GL.

The display scan driver 110 may be arranged in the non-display area NDA. Although the display scan driver 110 is illustrated as being arranged on one side (e.g., a left side) of the display panel 100, the present disclosure is not limited thereto. For example, the display scan driver 110 may also be arranged on both sides (e.g., opposite sides, e.g., left and right sides) of the display panel 100.

The display scan driver 110 may be electrically connected to the main driving circuit 200 through the gate control lines GCL. The display scan driver 110 receives a scan control signal from the main driving circuit 200, sequentially generates display scan signals in each horizontal line driving period according to the scan control signal, and sequentially supplies the display scan signals to the display scan lines GL. In one or more embodiments, the display scan driver 110 may sequentially generate emission control signals according to the scan control signal from the main driving circuit 200 and sequentially supply the emission control signals to the emission control lines VL.

The gate control lines GCL may extend from the main driving circuit 200 to the display scan driver 110 according to the position of the display scan driver 110. The gate control lines GCL may supply scan control signals received from the main driving circuit 200 to the display scan driver 110.

The light sensing scan driver 120 may be arranged in a different part of the non-display area NDA from the display scan driver 110. In FIG. 5, the light sensing scan driver 120 is arranged on the other side (e.g., the right side) of the display panel 100, but the present disclosure is not limited thereto. The light sensing scan driver 120 may be electrically connected to the main driving circuit 200 through the light sensing control lines SCL. The light sensing scan driver 120 receives a light sensing control signal from the main driving circuit 200 and sequentially generates reset control signals and sensing scan signals in each horizontal line driving period according to the light sensing control signal. Then, the reset control signals generated are sequentially supplied to the sensing reset lines REL. In one or more embodiments, the light sensing scan driver 120 may sequentially generate sensing scan signals according to the light sensing control signal from the main driving circuit 200 and sequentially supply the sensing scan signals to the light sensing scan lines FSL.

The light sensing control lines SCL may extend from the main driving circuit 200 to the light sensing scan driver 120 according to the position of the light sensing scan driver 120. The light sensing control lines SCL may supply light sensing control signals received from the main driving circuit 200 to the light sensing scan driver 120.

The sub-area SBA may include the main driving circuit 200, a display pad area DPA, and first and second touch pad areas TPA1 and TPA2. The display pad area DPA, the first touch pad area TPA1, and the second touch pad area TPA2 may be arranged at an edge of the sub-area SBA. The display pad area DPA, the first touch pad area TPA1, and the second touch pad area TPA2 may be electrically connected to the circuit board 300 using a low-resistance, high-reliability material such as an anisotropic conductive film or SAP.

The fan-out lines FOL may extend from the main driving circuit 200 to the display area DA. In one or more embodiments, the fan-out lines FOL are connected to the data lines DL so that data voltages received from the main driving circuit 200 can be supplied to the data lines DL, respectively.

The main driving circuit 200 may output signals and voltages for driving the display panel 100 to the fan-out lines FOL. The main driving circuit 200 may supply data voltages to the data lines DL through the fan-out lines FOL. The data voltages may be supplied to the display pixels SP and may determine luminances of the display pixels SP. The main driving circuit 200 may supply a scan control signal to the display scan driver 110 through a gate control line GCL. The main driving circuit 200 may generate digital video data according to touch coordinates based on touch coordinate data from the touch driving circuit 400 or may execute an application indicated by an icon displayed at coordinates of a user's touch.

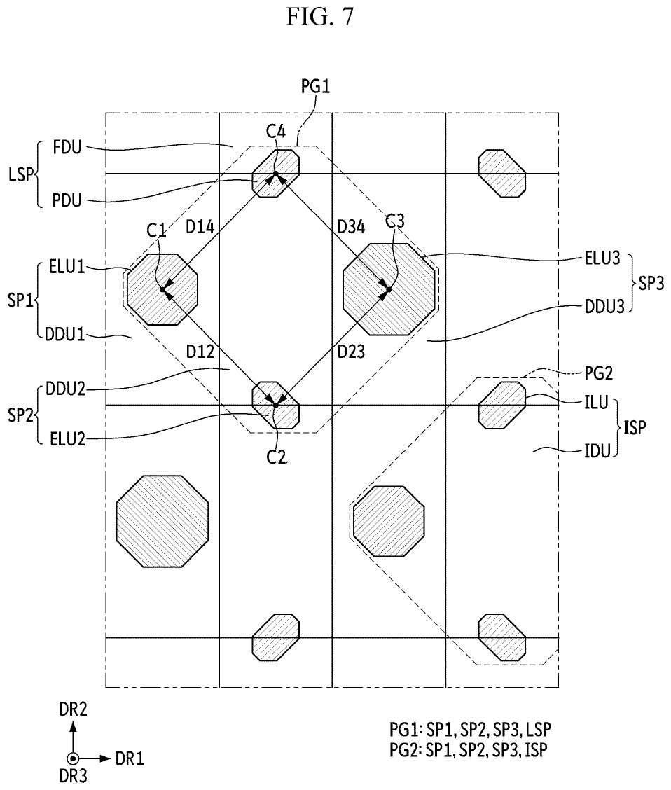

FIG. 7 is a layout view of a display area DA according to one or more embodiments of the present disclosure.

Referring to FIG. 7, the display area DA may include display pixels SP, infrared light emitting pixels ISP, and light sensing pixels LSP. Here, the display pixels SP may be divided into first display pixels SP1, second display pixels SP2, and third display pixels SP3.

A first display pixel SP1, a second display pixel SP2, a third display pixel SP3, and a light sensing pixel LSP may be defined as a first unit pixel PG1 (e.g., each first unit pixel PG1 may include a first display pixel SP1, a second display pixel SP2, a third display pixel SP3, and a light sensing pixel LSP). In one or more embodiments, a first display pixel SP1, a second display pixel SP2, a third display pixel SP3, and an infrared light emitting pixel ISP may be defined as a second unit pixel PG2 (e.g., each second unit pixel PG2 may include a first display pixel SP1, a second display pixel SP2, a third display pixel SP3, and an infrared light emitting pixel ISP).

Each of the first and second unit pixels PG1 and PG2 may be defined as the smallest unit of the display pixels that can display white. Each of the first unit pixels PG1 can sense light. The first unit pixels PG1 and the second unit pixels PG2 may be alternately arranged in a zigzag form in a plan view and may be arranged in a matrix form in any one diagonal direction. In one or more embodiments, the first unit pixels PG1 and the second unit pixels PG2 may be arranged in a matrix form by alternating in a horizontal or vertical stripe form in a plan view.

A first display pixel SP1 may include a first light emitting unit ELU1 emitting first light and a first pixel driving unit DDU1 for supplying a driving current to a light emitting element of the first light emitting unit ELU1. The first light may be light in a red wavelength band. For example, a main peak wavelength of the first light may be located at about 600 to 750 nm

A second display pixel SP2 may include a second light emitting unit ELU2 emitting second light and a second pixel driving unit DDU2 for supplying a driving current to a light emitting element of the second light emitting unit ELU2. The second light may be light in a blue wavelength band. For example, a main peak wavelength of the third light may be located at about 370 to 460 nm.

A third display pixel SP3 may include a third light emitting unit ELU3 emitting third light and a third pixel driving unit DDU3 for supplying a driving current to a light emitting element of the third light emitting unit ELU3. In one or more embodiments, the third light may be light in a blue wavelength band. In one or more embodiments, the third light may be light in a green wavelength band. For example, a main peak wavelength of the second light may be located at about 480 to 560 nm.

An infrared light emitting pixel ISP may include an infrared light emitting unit ILU emitting light in an infrared wavelength band and an infrared pixel driving unit IDU for supplying a driving current to a light emitting element of the infrared light emitting unit ILU. A main peak wavelength of infrared light may be located at about 750 nm to 1 mm.

A light sensing pixel LSP includes a light sensing unit PDU and a sensing driving unit FDU.

In a first unit pixel PG1, the first through third pixel driving units DDU1 through DDU3 may be arranged in a preset order in the first direction DR1. In one or more embodiments, any one of the first through third pixel driving units DDU1 through DDU3 may be arranged in the first direction DR1 relative to another adjacent pixel driving unit. In one or more embodiments, the sensing driving unit FDU may be arranged in the first direction DR1 relative to any one of the first through third pixel driving units DDU1 through DDU3. In one or more embodiments, the sensing driving unit FDU may be arranged in the second direction DR2 relative to any one of the first through third pixel driving units DDU1 through DDU3.

First pixel driving units DDU1 adjacent to each other in a data line direction may be arranged in the second direction DR2. Second pixel driving units DDU2 adjacent to each other in the data line direction may be arranged in the second direction DR2. Similarly, sensing driving units FDU adjacent to each other in the data line direction may all be arranged in the second direction DR2.

The first light emitting unit ELU1, the second light emitting unit ELU2, the third light emitting unit ELU3, the infrared light emitting unit ILU, and the light sensing unit PDU may have a quadrangular, octagonal, or rhombic planar shape. However, the present disclosure is not limited thereto. The first light emitting unit ELU1, the second light emitting unit ELU2, the third light emitting unit ELU3, the infrared light emitting unit ILU, and the light sensing unit PDU may also have a polygonal planar shape other than a quadrangle, an octagon, and a rhombus.

Due to the positions and planar shapes of the first light emitting unit ELU1, the second light emitting unit ELU2, the third light emitting unit ELU3 and the light sensing unit PDU, a distance D12 between a center C1 of the first light emitting unit ELU1 and a center C2 of the second light emitting unit ELU2 neighboring each other, a distance D23 between the center C2 of the second light emitting unit ELU2 and a center C3 of the third light emitting unit ELU3 neighboring each other, a distance D14 between the center C1 of the first light emitting unit ELU1 and a center C4 of the light sensing unit PDU neighboring each other, and a distance D34 between a center C4 of the second light emitting unit ELU2 (or, the center C4 of the light sensing unit PDU) and the center C3 of the third light emitting unit ELU3 may be substantially equal.

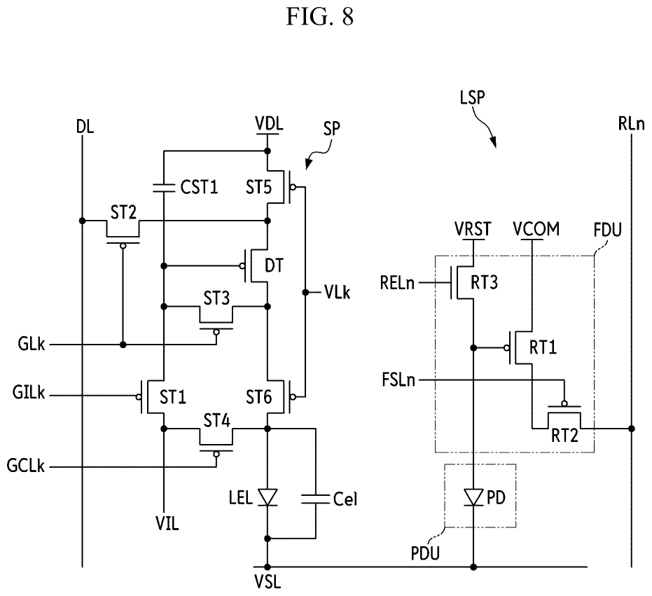

FIG. 8 is a circuit diagram of a display pixel SP and a light sensing pixel LSP according to one or more embodiments of the present disclosure.

Referring to FIG. 8, each display pixel SP according to one or more embodiments may be connected to a k^{th} display initialization line GILk, a k^{th} display scan line GLk, a k^{th} display control line GCLK, and a k^{th} emission control line VLK. In one or more embodiments, each display pixel SP may be connected to a first driving voltage line VDL to which a first driving voltage is supplied, a second driving voltage line VSL to which a second driving voltage is supplied, and a third driving voltage line VIL to which a third driving voltage is supplied. Here, letters k and n are used instead of numbers and are defined as positive integers excluding 0 (zero).

Each display pixel SP may include a light emitting unit ELU and a pixel driving unit DDU. The light emitting unit ELU may include a light emitting element LEL. The pixel driving unit DDU may include a driving transistor DT, switching elements, and a capacitor CST1. The switching elements include first through sixth transistors ST1 through ST6.

The driving transistor DT may include a gate electrode, a first electrode, and a second electrode. The driving transistor DT controls a drain-source current Ids (hereinafter, referred to as a “driving current”) flowing between the first electrode and the second electrode according to a data voltage applied to the gate electrode. The driving current Ids flowing through a channel of the driving transistor DT is proportional to the square of a difference between a voltage Vsg between the first electrode and the gate electrode of the driving transistor DT and a threshold voltage Vth as in Equation 1.

$$\begin{matrix}Equation 1 & \\ Ids = {k}^{′} \times {( V sg - V th )}^{2} , & ( 1 )\end{matrix}$$

In Equation 1, k′is a proportional coefficient determined by the structure and physical characteristics of a driving transistor, Vsg is a voltage between a first electrode and a gate electrode of the driving transistor, and Vth is a threshold voltage of the driving transistor.

The light emitting element LEL emits light according to the driving current Ids. As the driving current Ids increases, the amount of light emitted from the light emitting element LEL may increase.

The light emitting element LEL may be an organic light emitting diode including an organic light emitting layer arranged between an anode and a cathode. In one or more embodiments, the light emitting element LEL may be an inorganic light emitting element including an inorganic semiconductor arranged between an anode and a cathode. In one or more embodiments, the light emitting element LEL may be a quantum dot light emitting element including a quantum dot light emitting layer arranged between an anode and a cathode. In one or more embodiments, the light emitting element LEL may be a micro-light emitting element including a micro-light emitting diode arranged between an anode and a cathode.

The anode of the light emitting element LEL may be connected to a first electrode of the fourth transistor ST4 and a second electrode of the sixth transistor ST6, and the cathode may be connected to the second driving voltage line VSL. A parasitic capacitance CeI may be formed between the anode and the cathode of the light emitting element LEL.

The first transistor ST1 is turned on by an initialization scan signal of the k^{th} display initialization line GILk to connect the gate electrode of the driving transistor DT to the third driving voltage line VIL. Accordingly, the third driving voltage VINT of the third driving voltage line VIL may be applied to the gate electrode of the driving transistor DT. The first transistor ST1 may have a gate electrode connected to the k^{th} display initialization line GILk, a first electrode connected to the gate electrode of the driving transistor DT, and a second electrode connected to the third driving voltage line VIL.

The second transistor ST2 is turned on by a display scan signal of the k^{th} display scan line GLK to connect the first electrode of the driving transistor DT to a data line DL. Accordingly, a data voltage of the data line DL may be applied to the first electrode of the driving transistor DT. The second transistor ST2 may have a gate electrode connected to the k^{th} display scan line GLK, a first electrode connected to the first electrode of the driving transistor DT, and a second electrode connected to the data line DL.

The third transistor ST3 is turned on by the display scan signal of the k^{th} display scan line GLk to connect the gate electrode and the second electrode of the driving transistor DT. When the gate electrode and the second electrode of the driving transistor DT are connected, the driving transistor DT operates as a diode. The third transistor ST3 may have a gate electrode connected to the k^{th} display scan line GLk, a first electrode connected to the second electrode of the driving transistor DT, and a second electrode connected to the gate electrode of the driving transistor DT.

The fourth transistor ST4 is turned on by a display control signal of the k^{th} display control line GCLk to connect the anode of the light emitting element LEL to the third driving voltage line VIL. The third driving voltage of the third driving voltage line VIL may be applied to the anode of the light emitting element LEL. The fourth transistor ST4 may have a gate electrode connected to the k^{th} display control line GCLk, the first electrode connected to the anode of the light emitting element LEL, and a second electrode connected to the third driving voltage line VIL.

The fifth transistor ST5 is turned on by an emission control signal of the k^{th} emission control line VLK to connect the first electrode of the driving transistor DT to the first driving voltage line VDL. The fifth transistor ST5 may have a gate electrode connected to the k^{th} emission control line VLK, a first electrode connected to the first driving voltage line VDL, and a second electrode connected to the first electrode of the driving transistor DT.

The sixth transistor ST6 is arranged between the second electrode of the driving transistor DT and the anode of the light emitting element LEL. The sixth transistor ST6 is turned on by the emission control signal of the k^{th} emission control line VLK to connect the second electrode of the driving transistor DT to the anode of the light emitting element LEL. The sixth transistor ST6 may have a gate electrode connected to the k^{th} emission control line VLK, a first electrode connected to the second electrode of the driving transistor DT, and the second electrode connected to the anode of the light emitting element LEL.

When both the fifth transistor ST5 and the sixth transistor ST6 are turned on, the driving current Ids of the driving transistor DT according to the data voltage applied to the gate electrode of the driving transistor DT may flow to the light emitting element LEL.

The capacitor CST1 is formed between the gate electrode of the driving transistor DT and the first driving voltage line VDL. A first capacitor electrode of the capacitor CST1 may be connected to the gate electrode of the driving transistor DT, and a second capacitor electrode may be connected to the first driving voltage line VDL.

When the first electrode of each of the first through sixth transistors ST1 through ST6 and the driving transistor DT is a source electrode, the second electrode may be a drain electrode. In one or more embodiments, if (e.g., when) the first electrode of each of the first through sixth transistors ST1 through ST6 and the driving transistor DT is a drain electrode, the second electrode may be a source electrode.

An active layer of each of the first through sixth transistors ST1 through ST6 and the driving transistor DT may be made of any one of polysilicon, amorphous silicon, and/or an oxide semiconductor. Although embodiments in which the first through sixth transistors ST1 through ST6 and the driving transistor DT are formed as P-type (kind) metal-oxide-semiconductor field effect transistors (MOSFETs) has been mainly described in FIG. 8, the present disclosure is not limited thereto. For example, the first through sixth transistors ST1 through ST6 and the driving transistor DT may be formed as N-type (kind) MOSFETs. Alternatively, in one or more embodiments, at least one of the first through sixth transistors ST1 through ST6 may be formed as an N-type (kind) MOSFET.

Each light sensing pixel LSP is electrically connected to an n^{th} sensing reset line RELn, an n^{th} light sensing scan line FSLn, and an n^{th} light sensing line RLn. Each light sensing pixel LSP may be reset by a reset signal from the n^{th} sensing reset line RELn and may be to transmit a light sensing signal to the n^{th} light sensing line RLn in response to a sensing scan signal from the n^{th} light sensing scan line FSLn.

Each light sensing pixel LSP may be divided into a light sensing unit PDU including a photodetector PD and a sensing driving unit FDU including first through third sensing transistors RT1 through RT3 and a sensing capacitor. Here, the sensing capacitor may be formed in a parallel structure to the photodetector PD.

The first sensing transistor RT1 of the sensing driving unit FDU may allow a light sensing current to flow according to the voltage of the photodetector PD and the sensing capacitor. The amount of the light sensing current may vary according to the voltage applied to the photodetector PD and the sensing capacitor. A gate electrode of the first sensing transistor RT1 may be connected to a second electrode of the photodetector PD. A first electrode of the first sensing transistor RT1 may be connected to a common voltage source VCOM to which a common voltage is applied. A second electrode of the first sensing transistor RT1 may be connected to a first electrode of the second sensing transistor RT2.

The second sensing transistor RT2 may allow the sensing current of the first sensing transistor RT1 to flow to the n^{th} light sensing line RLn if (e.g., when) a sensing scan signal of a gate-on voltage is transmitted to the n^{th} light sensing scan line FSLn. In such embodiments, the n^{th} light sensing line RLn may be charged with a sensing voltage by the sensing current. The second sensing transistor RT2 may have a gate electrode connected to the n^{th} light sensing scan line FSLn, the first electrode connected to the second electrode of the first sensing transistor RT1, and a second electrode connected to the n^{th} light sensing line RLn.

The third sensing transistor RT3 may reset the voltage of the photodetector PD and the sensing capacitor to a reset voltage of a reset voltage source VRST if (e.g., when) a reset signal of a gate-on voltage is transmitted to the n^{th} sensing reset line RELn. The third sensing transistor RT3 may have a gate electrode connected to the sensing reset line REL, a first electrode connected to the reset voltage source VRST, and a second electrode connected to the second electrode of the photodetector PD.

In FIG. 8, embodiments in which the first sensing transistor RT1 and the second sensing transistor RT2 are formed as P-type (kind) MOSFETs and the third sensing transistor RT3 is formed as an N-type (kind) MOSFET has been mainly described. However, the present disclosure is not limited thereto, and the transistors may be formed as the same type (kind) or different types (kinds). In one or more embodiments, any one of the first electrode or the second electrode of each of the first sensing transistor RT1, the second sensing transistor RT2 and the third sensing transistor RT3 may be a source electrode, and the other may be a drain electrode.

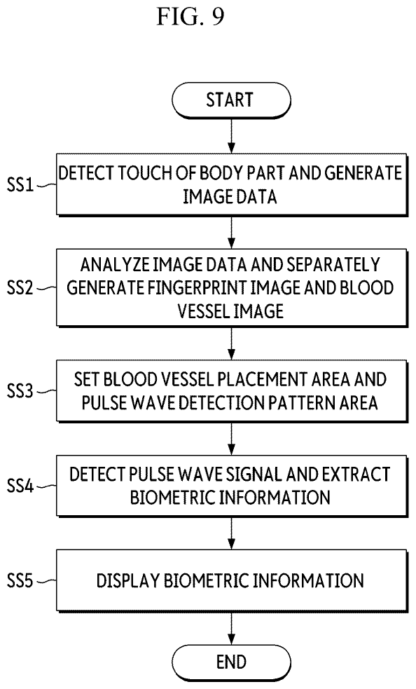

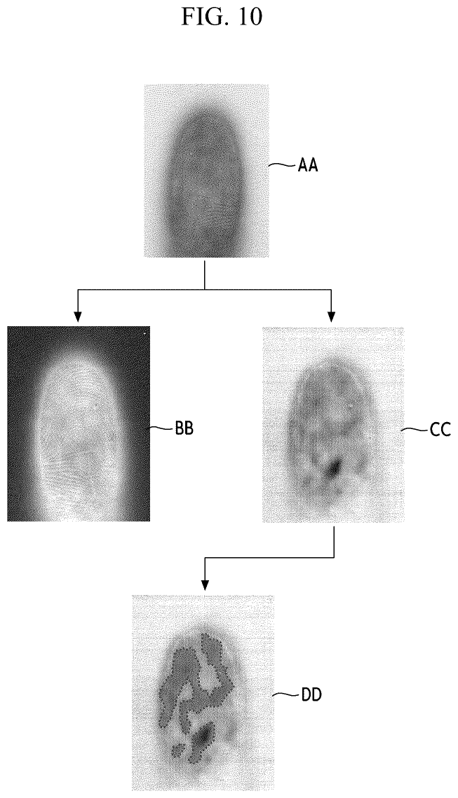

FIG. 9 is a flowchart illustrating a biometric information extraction process of the main driving circuit 200 according to one or more embodiments of the present disclosure. FIG. 10 shows images illustrating a pulse wave detection pattern area setting method of the main driving circuit 200 according to one or more embodiments of the present disclosure.

Referring to FIGS. 9 and 10, to detect a user's touch area during a user's touch position detection period, the main driving circuit 200 drives display pixels SP or infrared light emitting pixels ISP arranged in the biometric information measurement area FSA by controlling the scan signal output of the display scan driver 110 and the light sensing scan driver 120 by transmitting a scan control signal to the display scan driver 110 and the light sensing scan driver 120. In one or more embodiments, the main driving circuit 200 may detect light sensing signals of the biometric information measurement area FSA through light sensing pixels LSP. In such embodiments, the main driving circuit 200 may determine an emission color and luminance (or emission wavelength band) of the display pixels SP or the infrared light emitting pixels ISP included in the biometric information measurement area FSA.

For example, the main driving circuit 200 supplies a data voltage to at least one data line DL so that pixels emitting light of an emission color determined in real time or pixels emitting infrared light among the display pixels SP and the infrared light emitting pixels ISP in the biometric information measurement area FSA can emit light with a luminance or wavelength band determined in real time. In one or more embodiments, the main driving circuit 200 sequentially controls the driving of the display scan driver 110 and the light sensing scan driver 120. In one or more embodiments, the main driving circuit 200 receives a light sensing signal input through at least one of the light sensing lines ERL.

As illustrated in FIG. 10, the main driving circuit 200 converts light sensing signals of the light sensing pixels LSP received through the light sensing pixels LSP of the biometric information measurement area FSA into light sensing signal data, i.e., digital data (analog-to-digital conversion). Then, it generates image data AA for the biometric information measurement area FSA by arranging the light sensing signal data of the biometric information measurement area FSA (operation SS1). The main driving circuit 200 may also generate the image data AA for the biometric information measurement area FSA in units of multiple frames (e.g., each unit may be one frame) by continuously driving the display pixels SP or the infrared light emitting pixels ISP arranged in the biometric information measurement area FSA in units of at least one frame period and receiving the light sensing signals of the light sensing pixels LSP.

Next, the main driving circuit 200 separates and distinguishes fingerprint image data BB and blood vessel image data CC by comparing and analyzing a gray value or luminance value of the light sensing signal data of each light sensing pixel LSP of the image data AA with that of at least one adjacent light sensing pixel LSP (operation SS2). Here, the blood vessel image data CC includes light reflected by at least one blood vessel among veins and arteries, that is, light sensing signal data corresponding to a receiving area of light reflected by blood vessels.

Next, the main driving circuit 200 separates and distinguishes the light sensing signal data reflected by the blood vessels and a placement area of the light sensing signal data (i.e., a blood vessel placement area) from the blood vessel image data CC and sets a pulse wave detection pattern area corresponding to each placement area of the light sensing signal data (operation SS3). In other words, the main driving circuit 200 separates the blood vessel image data CC into light sensing signal data reflected by the blood vessels and location information regarding the placement of the blood vessels (i.e., the placement area), where data corresponding to each light sensing signal has a placement area, and then correlates and maps the light sensing signal data reflected by the blood vessels to the placement area in order to generate a map of the imaged blood vessels and sets the pulse wave detection pattern area to that area.

Here, the main driving circuit 200 generates pulse wave detection pattern image data DD including only gray values of the light sensing signal data by separating the light sensing signal data reflected by the blood vessels and the placement area of the light sensing signal data from the blood vessel image data CC. Then, the main driving circuit 200 sets a pulse wave detection pattern area in the display area DA or the biometric information measurement area FSA by detecting coordinate information about positions of the light sensing signal data from the pulse wave detection pattern image data DD including only the gray values of the light sensing signal data.

The main driving circuit 200 separately stores the pulse wave detection pattern image data DD and the coordinate information about the positions of the light sensing signal data in a built-in memory or the biometric information storage memory 500.

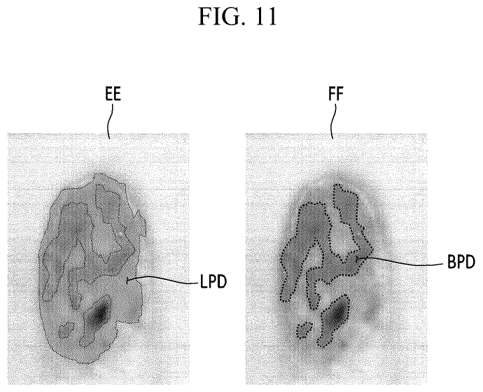

FIG. 11 are images illustrating a method of setting a light emitting area LPD and a light receiving area BPD separately in the pulse wave detection pattern area illustrated in FIG. 10, according to one or more embodiments of the present disclosure.

Referring to FIG. 11, the main driving circuit 200 may separately generate and store image data FF for a light receiving area BPD of the pulse wave detection pattern area and image data EE for a light emitting area LPD of the pulse wave detection pattern area.

For example, the main driving circuit 200 divides the pulse wave detection pattern area (where the pulse wave detection pattern area corresponds to the display area DA or the biometric information measurement area FSA) into the light receiving area BPD and the light emitting area LPD and calculates position coordinates of light sensing pixels LSP arranged to correspond to the light receiving area BPD. In one or more embodiments, the main driving circuit 200 calculates position coordinates of display pixels SP or infrared light emitting pixels ISP arranged to correspond to the light emitting area LPD.

The main driving circuit 200 separately stores, in the built-in memory or the biometric information storage memory 500, position coordinate information of the display pixels SP or the infrared light emitting pixels ISP arranged to correspond to the light emitting area LPD and position coordinate information of the light sensing pixels LSP arranged to correspond to the light receiving area BPD from the information from the pulse wave detection pattern area.

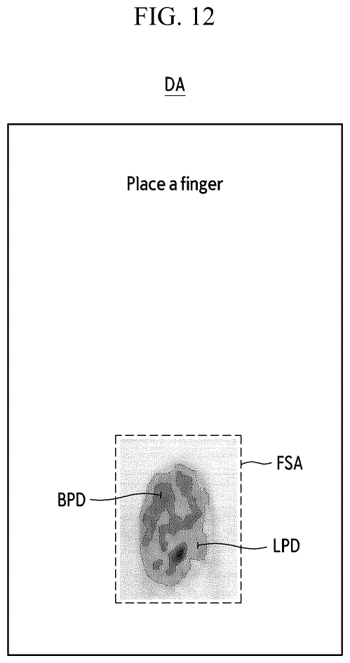

FIG. 12 is a diagram illustrating a reflected light sensing area and a pulse wave detection pattern area distinguished in a display area DA of one or more embodiments of the present disclosure.

Referring to FIG. 12, the main driving circuit 200 may induce a user to touch the display area DA and thus to provide the user's touch position and touch area by displaying the biometric information measurement area FSA in the display area DA during a user's biometric information detection period.

In one or more embodiments, the main driving circuit 200 drives the display pixels SP or the infrared light emitting pixels ISP arranged to correspond to the light emitting area LPD by controlling the scan signal output of the display scan driver 110 and the light sensing scan driver 120 and supplying a data signal of a preset size to the display pixels SP or the infrared light emitting pixels ISP arranged to correspond to the light emitting area LPD. In one or more embodiments, the main driving circuit 200 receives light sensing signals through the light sensing pixels LSP arranged to correspond to the light receiving area BPD by driving the light sensing pixels LSP arranged to correspond to the light receiving area BPD.

The main driving circuit 200 may generate a pulse wave signal, which reflects blood changes according to heartbeat, using the light sensing signals received through the light sensing pixels LSP arranged to correspond to the light receiving area BPD and thus may measure a user's biometric information such as blood pressure according to the size and change cycle of the pulse wave signal (operation SS4, as shown in FIG. 9).

Next, the main driving circuit 200 may generate result image data based on the results of measuring biometric information in real time and display the result image data as an image on the display panel 100.

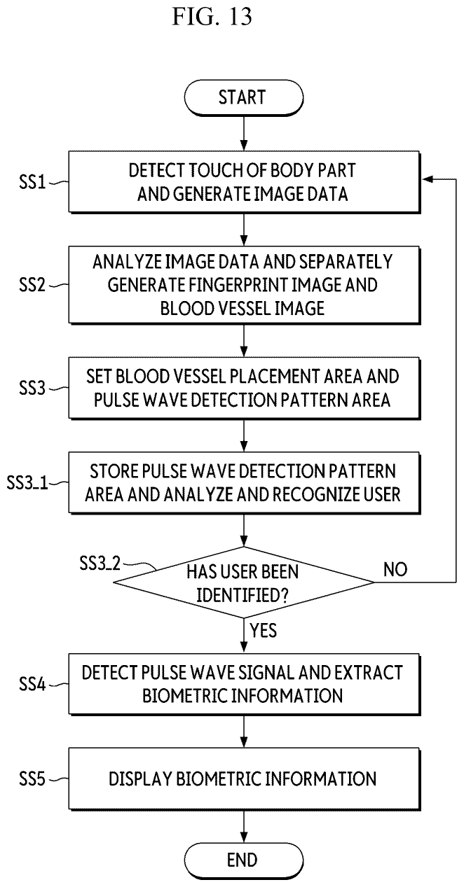

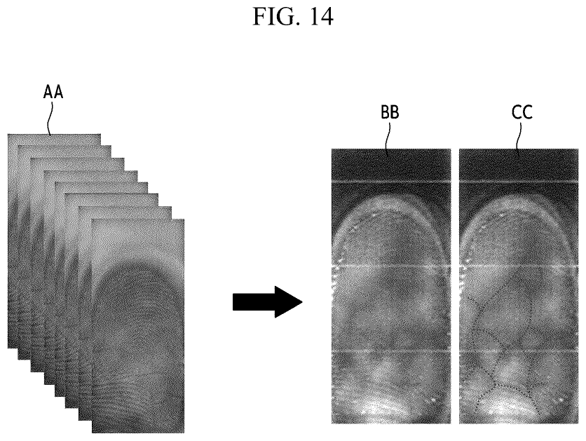

FIG. 13 is a flowchart illustrating a biometric information extraction process of the main driving circuit 200 according to one or more embodiments of the present disclosure. FIG. 14 shows images illustrating a pulse wave detection pattern area setting method of the main driving circuit 200 according to one or more embodiments of the present disclosure.

Referring to FIGS. 13 and 14, to detect a user's touch area during a user's touch position detection period, the main driving circuit 200 drives display pixels SP or infrared light emitting pixels ISP arranged in the biometric information measurement area FSA by controlling the scan signal output of the display scan driver 110 and the light sensing scan driver 120. In one or more embodiments, the main driving circuit 200 may detect light sensing signals of the biometric information measurement area FSA through light sensing pixels LSP.

As illustrated in FIG. 14, the main driving circuit 200 converts the light sensing signals of the light sensing pixels LSP received through the light sensing pixels LSP of the biometric information measurement area FSA into light sensing signal data (analog-to-digital conversion). Then, it generates image data AA for the biometric information measurement area FSA by arranging the light sensing signal data of the biometric information measurement area FSA (operation SS1).

The main driving circuit 200 may also generate the image data AA for the biometric information measurement area FSA in units of multiple frames (e.g., each unit may be one frame) by continuously driving the display pixels SP or the infrared light emitting pixels ISP arranged in the biometric information measurement area FSA in units of at least one frame period and receiving the light sensing signals of the light sensing pixels LSP.

Next, the main driving circuit 200 separates and distinguishes fingerprint image data BB and blood vessel image data CC by comparing and analyzing a gray value or luminance value of the light sensing signal data of each infrared light emitting pixel ISP of the image data AA with that of at least one adjacent light sensing pixel LSP (operation SS2).

Next, the main driving circuit 200 separates and distinguishes light sensing signal data reflected by blood vessels and a placement area of the light sensing signal data (i.e., a blood vessel placement area) from the blood vessel image data CC and sets a pulse wave detection pattern area corresponding to each placement area of the light sensing signal data (operation SS3).

Here, the main driving circuit 200 generates pulse wave detection pattern image data DD including only gray values of the light sensing signal data by separating the light sensing signal data reflected by the blood vessels and the placement area of the light sensing signal data from the blood vessel image data CC. Then, the main driving circuit 200 sets a pulse wave detection pattern area in the display area DA or the biometric information measurement area FSA by detecting coordinate information about positions of the light sensing signal data from the pulse wave detection pattern image data DD including only the gray values of the light sensing signal data.

The main driving circuit 200 separately stores the pulse wave detection pattern image data DD and the coordinate information about the positions of the light sensing signal data in a built-in memory or the biometric information storage memory 500.

FIG. 15 is a diagram illustrating a user recognition method using a pulse wave detection pattern area,, according to one or more embodiments of the present disclosure.

Referring to FIG. 15, the main driving circuit 200 compares the pulse wave detection pattern image data DD and the coordinate information about the positions of the light sensing signal data with pulse wave detection pattern image data DD and coordinate information about positions of light sensing signal data for users which are stored separately in the built-in memory or the biometric information storage memory 500 and analyzes and recognizes a user based on whether there is a match according to the comparison result (operation SS3_1).

The main driving circuit 200 determines whether to proceed with a biometric information measurement process based on the result of user analysis and recognition (operation SS3_2).

The main driving circuit 200 may separately generate and store image data FF for a light receiving area BPD of the pulse wave detection pattern area and image data EE for a light emitting area LPD of the pulse wave detection pattern area.

For example, the main driving circuit 200 divides the pulse wave detection pattern area set to correspond to the display area DA or the biometric information measurement area FSA into the light receiving area BPD and the light emitting area LPD and calculates position coordinates of light sensing pixels LSP arranged to correspond to the light receiving area BPD. In one or more embodiments, the main driving circuit 200 calculates position coordinates of display pixels SP or infrared light emitting pixels ISP arranged to correspond to the light emitting area LPD.

The main driving circuit 200 separately stores, in the built-in memory or the biometric information storage memory 500 (see, e.g., FIG. 1), position coordinate information of the display pixels SP or the infrared light emitting pixels ISP arranged to correspond to the light emitting area LPD and position coordinate information of the light sensing pixels LSP arranged to correspond to the light receiving area BPD among information about the pulse wave detection pattern area.

The main driving circuit 200 may compare the position coordinates of the light sensing pixels LSP arranged to correspond to the light receiving area BPD and the position coordinates of the display pixels SP or the infrared light emitting pixels ISP arranged to correspond to the light emitting area LPD with position coordinates of light sensing pixels LSP and position coordinates of infrared light emitting pixels ISP for each user which are stored separately in the built-in memory or the biometric information storage memory 500 and may analyze and recognize a user based on whether there is a match according to the comparison result.

The main driving circuit 200 may induce a user to touch the display area DA and thus to provide the user's touch position and touch area by displaying the biometric information measurement area FSA in the display area DA during a user's biometric information detection period.

In one or more embodiments, the main driving circuit 200 drives the display pixels SP or the infrared light emitting pixels ISP arranged to correspond to the light emitting area LPD by controlling the scan signal output of the display scan driver 110 and the light sensing scan driver 120 and supplying a data signal of a preset size to the display pixels SP or the infrared light emitting pixels ISP arranged to correspond to the light emitting area LPD. In one or more embodiments, the main driving circuit 200 receives light sensing signals through the light sensing pixels LSP arranged to correspond to the light receiving area BPD by driving the light sensing pixels LSP arranged to correspond to the light receiving area BPD.

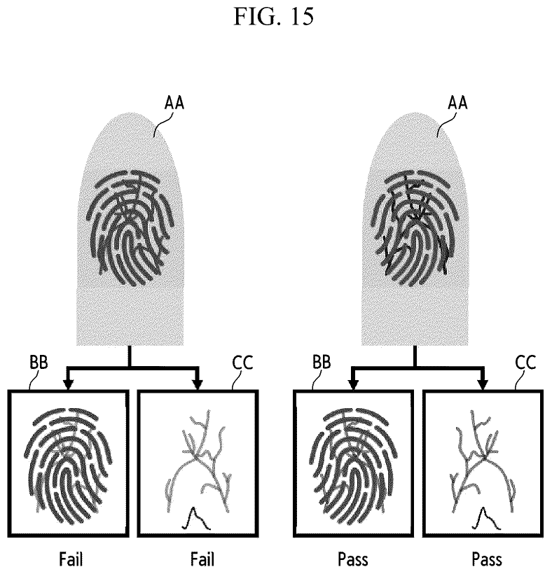

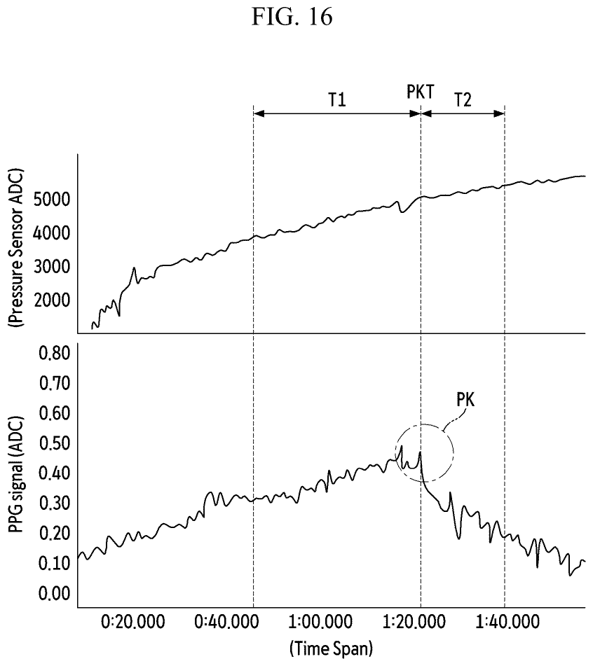

FIG. 16 is a graph for explaining a method of calculating blood pressure information from among the biometric information according to one or more embodiments of the present disclosure.

The main driving circuit 200 may generate a pulse wave signal, which reflects blood changes according to heartbeat, using light sensing signals received through the light sensing pixels LSP arranged to correspond to the light receiving area BPD and thus may measure a user's biometric information such as blood pressure according to the size and change cycle of the pulse wave signal (operation SS4).

For example, during the systole of the heart, the blood ejected from the left ventricle of the heart moves to peripheral tissues, thus increasing blood volume on the arterial side. In addition, during the systole of the heart, red blood cells carry more oxyhemoglobin to the peripheral tissues. During the diastole of the heart, there is partial suction of blood from the peripheral tissues toward the heart. When light is irradiated to peripheral blood vessels, the irradiated light is absorbed by the peripheral tissues. Light absorbance is dependent on hematocrit and blood volume. The light absorbance may have a maximum value during the systole of the heart and a minimum value during the diastole of the heart. Therefore, light detected by a photodetector PD may be the least during the systole of the heart and the most during the diastole of the heart.

In one or more embodiments, if (e.g., when) a user places a finger on the display panel 100 and then removes the finger in a blood pressure measurement mode, the pressure (contact pressure) applied to the pressure sensing unit PSU may gradually increase, reach a maximum value, and then gradually decrease. If the contact pressure increases, the blood vessels may shrink, causing the blood flow to decrease or become zero. If the contact pressure decreases, the blood vessels may expand, causing the blood to flow again. If the contact pressure decreases further, the blood flow increases further. Therefore, a change in the amount of light detected by a light sensing pixel LSP may be proportional to a change in blood flow. Accordingly, the main driving circuit 200 generates pulse wave signals PPG according to the pressure applied by a user based on a pressure data value calculated and digitally converted by the analog-to-digital conversion of the pressure sensing unit PSU and an optical signal according to the amount of light detected by a photodetector PD (PPG signal ratio). The pulse wave signals PPG may have a waveform that vibrates according to the heartbeat cycle.

The main driving circuit 200 may estimate blood pressures of the blood vessels of the finger F based on time differences between time points PKT corresponding to peaks PK of the generated pulse wave signals PPG and time points corresponding to peaks of a filtered pulse wave. For example, the main driving circuit 200 may generate pulse wave signals during preset periods T1 and T2 before and after the time points PKT corresponding to the peaks PK of a generated pulse wave signal and detect blood pressures according to differences between the pulse wave signals. Among the estimated blood pressures, maximum blood pressure may be calculated as systolic blood pressure, and minimum blood pressure may be calculated as diastolic blood pressure. In one or more embodiments, other blood pressures, such as average blood pressure, can be calculated using the estimated blood pressures.

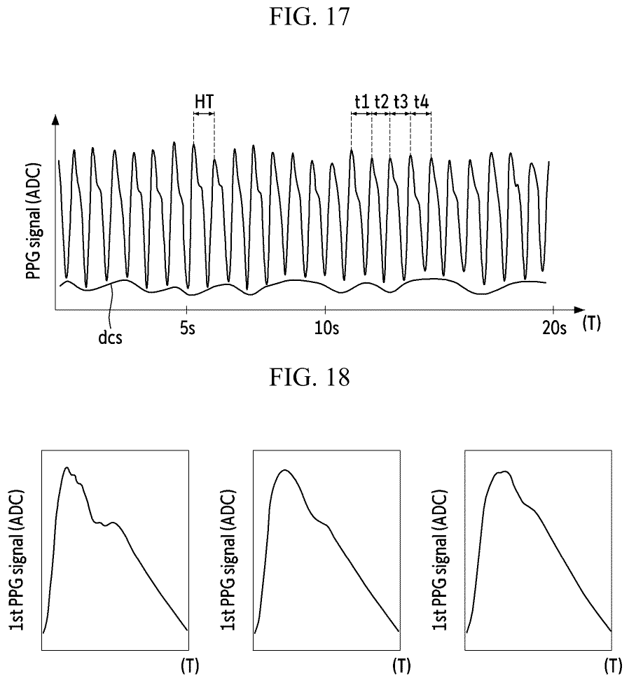

FIG. 17 is a graph for explaining a method of calculating information about heartbeat and respiration from among the biometric information, according to one or more embodiments of the present disclosure.

Referring to FIG. 17, the main driving circuit 200 samples pulse wave signals during preset sampling periods before and after time points PKT corresponding to peaks PK of a pulse wave signal and detects a generation cycle HT of high pulses for the sampled pulse wave signals PPG. In one or more embodiments, the main driving circuit 200 may detect biometric information about heartbeat cycle and heart rate (HR) by counting the number of high pulses generated for each preset reference period (e.g., 60 seconds) for the sampled pulse wave signals PPG.

In one or more embodiments, the main driving circuit 200 detects heart rate variability (HRV) according to a heartbeat cycle variation rate by detecting the generation cycle HT and generation cycle variation (t1 through t4) of high pulses for the peaks PK of the sampled pulse wave signals PPG for each preset reference period.

In one or more embodiments, the main driving circuit 200 sequentially detects a generation cycle of low pulses and size values of the low pulses for the sampled pulse wave signals PPG. In one or more embodiments, it may detect a user's breathing change state and respiration rate by detecting a change cycle of the size values dos of the low pulses for each preset reference period (e.g., 60 seconds). Here, a rising cycle of the size values dos of the low pulses and a falling cycle of the size values des of the low pulses may be analyzed to detect the user's breathing change state and respiration rate (RR) at the rising and falling cycles of the size values des of the low pulses.

FIG. 18 is a graph for explaining a method of calculating information about blood vessel elasticity from among the biometric information, according to one or more embodiments of the present disclosure.

Referring to FIG. 18, the main driving circuit 200 may set and derive blood vessel elasticity (BVE) by enlarging and analyzing the high pulse fluctuation form of sampled pulse wave signals PPG.

When the blood flow increases due to the heartbeat, a pulse wave signal changes to a high pulse form. When the blood flow decreases, the pulse wave signal changes again to a low pulse form. If the blood flow changes rapidly due to the shape of blood vessels during a period of increasing or decreasing blood flow, the change in the blood flow may quickly or slowly calm down depending on the elasticity of the blood vessels. Accordingly, the main driving circuit 200 enlarges and analyzes the high pulse fluctuation form of the pulse wave signals PPG and sets and derives the blood vessel elasticity as a value corresponding to the fluctuation size of the high pulses.

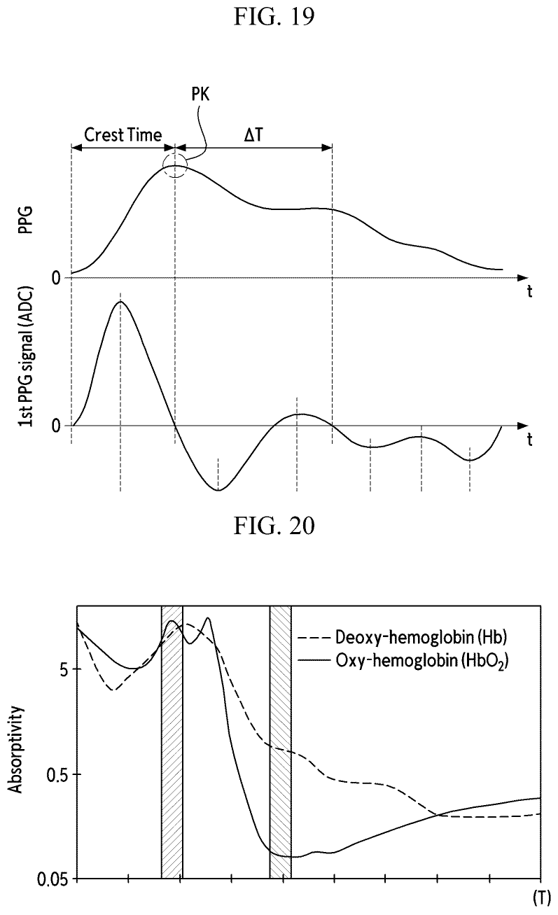

FIG. 19 is a graph for explaining a method of calculating information about cardiovascular disease from among the biometric information, according to one or more embodiments of the present disclosure.

Referring to FIG. 19, the main driving circuit 200 may set and derive an evaluation score for cardiovascular disease (or a cardiovascular health analysis result score) by differentiating and enlarging and analyzing the high pulse fluctuation form of sampled pulse wave signals PPG. For example, the main driving circuit 200 detects a period (Crest Time) during which the pulse wave signals PPG reach a peak PK in the form of a high pulse and a change in time (ΔT) during which the pulse wave signals PPG fall relative to the period (Crest Time) during which the pulse wave signals PPG reach the peak PK in the form of a high pulse. The longer the period (Crest Time) during which the pulse wave signals PPG reach the peak PK in the form of a high pulse, the greater the risk of heart disease. Accordingly, the main driving circuit 200 may set and derive the evaluation score for cardiovascular disease (or the cardiovascular health analysis result score) in inverse proportion to the period (Crest Time) during which the pulse wave signals PPG reach the peak PK in the form of a high pulse.

FIG. 20 is a graph for explaining a method of calculating information about oxygen saturation from among the biometric information, according to one or more embodiments of the present disclosure.

Referring to FIG. 20, during the systole of the heart, red blood cells carry more oxyhemoglobin to peripheral tissues. In contrast, during the diastole of the heart, there is partial suction of blood from the peripheral tissues toward the heart. Using this, the main driving circuit 200 detects a deoxy-hemoglobin (Hb) level based on a change in the size of pulse wave signals PPG detected by green light and detects an oxy-hemoglobin (HbO₂) level based on a change in the size of the pulse wave signals PPG detected by red light.

The main driving circuit 200 may detect oxygen saturation (SpO₂) using Equation 2.

$${SpO}_{2} = {HbO}_{2} / ( {HbO}_{2} + Hb ) \qquad ( 2 )$$

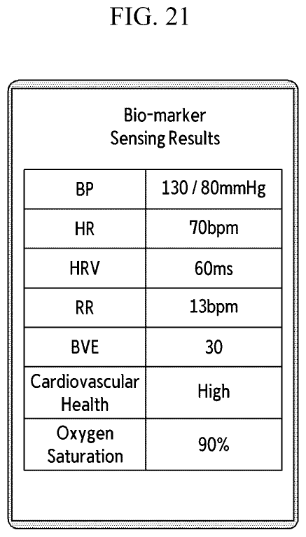

FIG. 21 illustrates the results of measuring biometric information using a display device, according to one or more embodiments of the present disclosure.

Referring to FIG. 21, the main driving circuit 200 may generate resultant image data based on the results of measuring biometric information in real time and display the resultant image data as an image on the display panel 100.

For example, the main driving circuit 200 may display biometric information such as blood pressure (BP), heart rate (HR), heart rate variability (HRV), respiratory rate (RR), blood vessel elasticity (BVE), cardiovascular disease (or cardiovascular health analysis result score), and/or oxygen saturation (SpO₂) on an application program screen.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Further, the use of “may” when describing embodiments of the present disclosure refers to “one or more embodiments of the present disclosure.”

As used herein, the term “substantially,” “about,” and similar terms are used as terms of approximation and not as terms of degree, and are intended to account for the inherent deviations in measured or calculated values that would be recognized by those of ordinary skill in the art. “Substantially” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “substantially” may mean within one or more standard deviations, or within ±30%, 20%, 10%, 5% of the stated value.

The light emitting device, electronic apparatus or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

A person of ordinary skill in the art, in view of the present disclosure in its entirety, would appreciate that each suitable feature of the various embodiments of the present disclosure may be combined or combined with each other, partially or entirely, and may be technically interlocked and operated in various suitable ways, and each embodiment may be implemented independently of each other or in conjunction with each other in any suitable manner unless otherwise stated or implied.

It will be understood that descriptions of features or aspects within each embodiment should typically be considered as available for other similar features or aspects in other embodiments, unless otherwise described. Thus, as would be apparent to one of ordinary skill in the art, features, characteristics, and/or elements described in connection with a particular embodiment may be used singly or in combination with features, characteristics, and/or elements described in connection with other embodiments unless otherwise specifically indicated. It is to be understood that the foregoing is an illustration of various example embodiments and is not to be construed as limited to the specific embodiments disclosed herein, and that various modifications to the disclosed embodiments, as well as other example embodiments, are intended to be included within the spirit and scope of the present disclosure as defined in the appended claims, and their equivalents.

What is claimed is:

1. A display device comprising:
a display panel;
display pixels in a display area of the display panel;
light sensing pixels in the display area together with the display pixels;
infrared light emitting pixels in the display area together with the display pixels;
a display scan driver to drive the display pixels and the light sensing pixels to emit light;
a light sensing scan driver to drive the light sensing pixels to detect light; and
a main driving circuit to detect pulse wave signals of a user utilizing light sensing signals received through the light sensing pixels and to measure biometric information,
wherein the main driving circuit is configured to separate and to generate blood vessel image data from image data for a touch area during a user's touch position detection period and to receive the light sensing signals of a light receiving area by distinguishing the light receiving area according to the blood vessel image data in the display area during a user's biometric information detection period.

2. The display device of claim 1, wherein the main driving circuit is configured to display a biometric information measurement area in the display area as an application program screen, to drive the display pixels or the infrared light emitting pixels in the biometric information measurement area by controlling a scan signal output of the display scan driver and the light sensing scan driver, and to receive the light sensing signals through the light sensing pixels of the biometric information measurement area.

3. The display device of claim 2, wherein the main driving circuit is configured to convert the light sensing signals of the light sensing pixels received through the light sensing pixels of the biometric information measurement area into digital light sensing signal data and to generate image data for the biometric information measurement area in units of multiple frames by arranging the light sensing signal data.

4. The display device of claim 3, wherein the main driving circuit is configured to separate and to divide the image data into fingerprint image data and blood vessel image data by comparing and analyzing a gray value or luminance value of the light sensing signal data of each light sensing pixel of the light sensing pixels included in the image data with that of at least one adjacent light sensing pixel of the light sensing pixels.

5. The display device of claim 4, wherein the main driving circuit is configured to separate and to distinguish light sensing signal data reflected by blood vessels and a placement area of the light sensing signal data from the blood vessel image data and to generate a pulse wave detection pattern area comprising only gray values of the light sensing signal data and corresponding to the placement area of the light sensing signal data and pulse wave detection pattern image data corresponding to the pulse wave detection pattern area.

6. The display device of claim 5, wherein the main driving circuit is configured to set the pulse wave detection pattern area in the display area or the biometric information measurement area by detecting coordinate information about positions of the light sensing signal data from the pulse wave detection pattern image data and to separately store the pulse wave detection pattern image data and the coordinate information about the positions of the light sensing signal data in a built-in memory or a biometric information storage memory.

7. The display device of claim 6, wherein the main driving circuit is configured to compare the pulse wave detection pattern image data and the coordinate information about the positions of the light sensing signal data with pulse wave detection pattern image data and coordinate information about positions of light sensing signal data for users which are stored separately in the built-in memory or the biometric information storage memory and to analyze and recognize a user based on whether there is a match according to a comparison result.

8. The display device of claim 6, wherein the main driving circuit is configured to divide the pulse wave detection pattern area into a light receiving area and a light emitting area, to calculate position coordinates of the light sensing pixels to correspond to the light receiving area, to calculate position coordinates of the display pixels or the infrared light emitting pixels to correspond to the light emitting area, and to separately store position coordinate information of the display pixels or the infrared light emitting pixels to correspond to the light emitting area and position coordinate information of the light sensing pixels to correspond to the light receiving area in the built-in memory or the biometric information storage memory.

9. The display device of claim 8, wherein, during the user's biometric information detection period, the main driving circuit drives the display pixels or the infrared light emitting pixels to correspond to the light emitting area, receives light sensing signals through the light sensing pixels to correspond to the light receiving area, generates a pulse wave signal, which reflects blood changes according to heartbeat, utilizing the light sensing signals received through the light sensing pixels to correspond to the light receiving area, and measures biometric information.

10. The display device of claim 8, wherein the main driving circuit is configured to compare the position coordinates of the light sensing pixels corresponding to the light receiving area and the position coordinates of the display pixels or the infrared light emitting pixels corresponding to the light emitting area with position coordinates of light sensing pixels and position coordinates of infrared light emitting pixels for each user which are stored separately in the built-in memory or the biometric information storage memory and to analyze and recognize a user based on whether there is a match according to a comparison result.

11. A display device comprising:
a display panel;
display pixels in a display area of the display panel;
light sensing pixels in the display area together with the display pixels;
infrared light emitting pixels in the display area together with the display pixels;
a display scan driver to drive the display pixels and the light sensing pixels to emit light;
a light sensing scan driver to drive the light sensing pixels to detect light; and
a main driving circuit to detect pulse wave signals of a user utilizing light sensing signals received through the light sensing pixels and to measure biometric information,
wherein the main driving circuit is configured to display a biometric information measurement area as an application program screen in the display area, to drive the display pixels or the infrared light emitting pixels in the biometric information measurement area by controlling a scan signal output of the display scan driver and the light sensing scan driver, and to receive the light sensing signals through the light sensing pixels of the biometric information measurement area.

12. The display device of claim 11, wherein the main driving circuit is configured to separate and to generate blood vessel image data from image data for a touch area during a user's touch position detection period and receive the light sensing signals of a light receiving area by distinguishing the light receiving area according to the blood vessel image data in the display area during a user's biometric information detection period.

13. The display device of claim 12, wherein the main driving circuit is configured to convert the light sensing signals of the light sensing pixels received through the light sensing pixels of the biometric information measurement area into digital light sensing signal data, to generate image data for the biometric information measurement area in units of multiple frames by arranging the light sensing signal data, and to separate and divide the image data into fingerprint image data and blood vessel image data by comparing and analyzing a gray value or luminance value of the light sensing signal data of each light sensing pixel included in the image data with that of at least one adjacent light sensing pixel.

14. The display device of claim 13, wherein the main driving circuit is configured to separate and to distinguish light sensing signal data reflected by blood vessels and a placement area of the light sensing signal data from the blood vessel image data and to generate a pulse wave detection pattern area comprising only gray values of the light sensing signal data and corresponding to the placement area of the light sensing signal data and pulse wave detection pattern image data corresponding to the pulse wave detection pattern area.

15. The display device of claim 14, wherein the main driving circuit is configured to set the pulse wave detection pattern area in the display area or the biometric information measurement area by detecting coordinate information about positions of the light sensing signal data from the pulse wave detection pattern image data and to separately store the pulse wave detection pattern image data and the coordinate information about the positions of the light sensing signal data in a built-in memory or a biometric information storage memory.

16. The display device of claim 15, wherein the main driving circuit is configured to compare the pulse wave detection pattern image data and the coordinate information about the positions of the light sensing signal data with pulse wave detection pattern image data and coordinate information about positions of light sensing signal data for users which are stored separately in the built-in memory or the biometric information storage memory and to analyze and recognize a user based on whether there is a match according to a comparison result.

17. The display device of claim 16, wherein the main driving circuit is configured to divide the pulse wave detection pattern area into a light receiving area and a light emitting area, to calculate position coordinates of the light sensing pixels to correspond to the light receiving area, to calculate position coordinates of the display pixels or the infrared light emitting pixels to correspond to the light emitting area, and to separately store position coordinate information of the display pixels or the infrared light emitting pixels to correspond to the light emitting area and position coordinate information of the light sensing pixels to correspond to the light receiving area in the built-in memory or the biometric information storage memory.

18. The display device of claim 17, wherein the main driving circuit is configured to compare the position coordinates of the light sensing pixels corresponding to the light receiving area and the position coordinates of the display pixels or the infrared light emitting pixels corresponding to the light emitting area with position coordinates of light sensing pixels and position coordinates of infrared light emitting pixels for each user which are stored separately in the built-in memory or the biometric information storage memory and to analyze and recognize a user based on whether there is a match according to a comparison result.

19. An electronic device comprising:
a display device comprising
a display panel;
display pixels in a display area of the display panel;
light sensing pixels in the display area together with the display pixels;
infrared light emitting pixels in the display area together with the display pixels;
a display scan driver to drive the display pixels and the light sensing pixels to emit light;
a light sensing scan driver to drive the light sensing pixels to detect light; and
a main driving circuit to detect pulse wave signals of a user utilizing light sensing signals received through the light sensing pixels and to measure biometric information,
wherein the main driving circuit is configured to separate and to generate blood vessel image data from image data for a touch area during a user's touch position detection period and to receive the light sensing signals of a light receiving area by distinguishing the light receiving area according to the blood vessel image data in the display area during a user's biometric information detection period.

20. The electronic device of claim 19, wherein the electronic device comprises a mobile phone, a smartphone, a tablet personal computer, a mobile communication terminal, an electronic notebook, an e-book, a portable multimedia player, a navigation system, an ultramobile personal computer, a television, a laptop, a monitor, a billboard, a smart watch, a watch phone, a glasses-type display, a head mounted display, or a car display.
